(19) United States
(12) Patent Application Publication
(10) Pub. No.: US 2004/0089293 A1
(43) Pub. Date: May 13, 2004

(54) Medicament dispenser

(76) Inventors: Paul Johnson, Ware (GB); David Michael Ottolangui, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/399,009

(22) PCT Filed: Sep. 26, 2001
(86) PCT No.: PCT/EP01/11098

(30) Foreign Application Priority Data
Oct. 13, 2000 (GB) 0025092.8
Jul. 11, 2001 (GB) 0116891.3

(51) Int. Cl. A61M 11/00

(52) U.S. Cl. 128/200.23

(57) ABSTRACT

A dispenser for dispensing a medicament in a fluid propellant comprising: a) a canister for housing the medicament; b) a drug-dispensing valve made substantially of metal; and c) moisture absorbing means for absorbing moisture, is disclosed.

The present invention relates to medicament dispensers. More especially, the invention relates to a metered dose inhaler and components thereof that substantially alleviate or prevent moisture build-up therein. The invention also relates to a method for reducing moisture build-up inside a metered dose inhaler.

Drugs for treating respiratory and nasal disorders are frequently administered in aerosol formulations through the mouth or nose. One widely used method for dispensing such aerosol drug formulations involves formulating the drug as a suspension or a solution in a liquefied gas propellant. The suspension/solution is stored in a sealed canister capable of withstanding the pressure required to maintain the propellant as a liquid. The suspension/solution is dispersed by activation of a valve affixed to the canister.

Containers for aerosol formulations commonly comprise a vial body (canister), a crimped cap covering the mouth of the canister, a drug metering valve situated in the cap, a metering chamber and a suitable channelling device in to which the canister is fitted.

A drug metering valve generally comprises a metering chamber that is of a set volume and is designed to administer per actuation an accurate predetermined dose of medicament. As the suspension is forced from the canister through the dose-metering valve by the high vapour pressure of the propellant, the propellant rapidly vaporises leaving a fast moving cloud of very fine particles of the drug formulation. This cloud of particles is directed into the nose or mouth of the patient by a channelling device such as a cylinder or cone-like passage through which medicament may be delivered for the filled canister via the valve to the nose or mouth of a patient, e.g. via a mouthpiece actuator. Concurrently with the activation of the aerosol dose-metering valve, the patent inhales the drug particles into the lungs or nasal cavity. Systems of dispensing drugs in this way are known as “metered dose inhalers” (MDI's). See Peter Byron, Respiratory Drug Delivery, CRC Press, Boca Raton, Fla. (1990) for a general background on this form of therapy.

Patients often rely on medication delivered by MDI's for rapid treatment of respiratory disorders that are debilitating and in some cases even life threatening. Therefore, it is essential that the prescribed dose of aerosol medication delivered to the patient consistently meets the specifications claimed by the manufacturer and complies with the requirements of the FDA and other regulatory authorities. That is, every dose in the can must be the same within close tolerances.

To obtain regulatory approval pharmaceutical aerosol formulation products must meet strict specifications. One parameter for which a specification is usually set is the fine particle mass (FPM). This is a means of evaluating the amount of drug substance which has the potential to reach the inner lungs, ie the small bronchioles and alveoli, based on the amount of drug particles within a certain range, usually less than 5 microns. The FPM of an actuation from an MDI can be calculated based on, for example, the sum of the amount of drug substances deposited on stages 3, 4 and 5 of an Anderson Cascade Impaction stack as determined by standard HPLC analysis; the methodology and apparatus is described in Experimental Detail, infra. It is important that the FPM of the pharmaceutical aerosol formulation, for all the doses dispensed from the MDI, is within the specification set, even after the MDI has been stored for a prolonged period.

For environmental reasons, there has been a move to replace chlorofluorocarbons (CFCs) (also simply known as “fluorocarbons”) such as P11, P114 and P12 with hydrofluoroalkane propellants such as HFA-134a and HFA-227.

A problem which can exist under certain conditions with the latest range of CFC-free, HFA-propellant based formulations is moisture ingress into the aerosol container through the seal of the container or the drug metering valve.

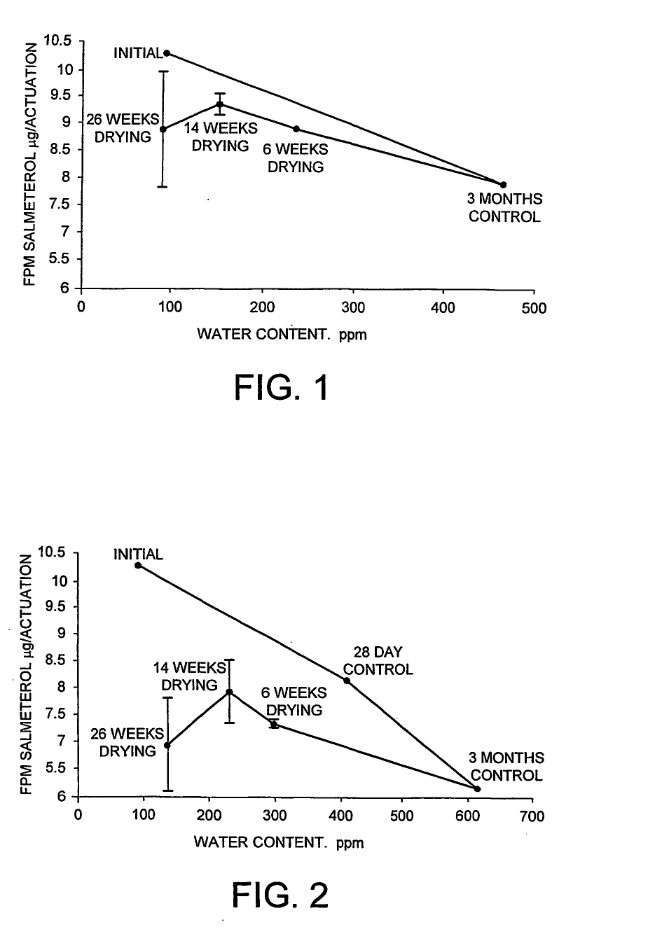

Studies have shown that reducing the moisture content of a metered dose inhaler can result in an increase in the FPM. FIGS. 1 and 2 illustrate the relationship between water content and FPM for metered dose inhalers containing samples of Salmeterol (Serevent). Control values for FPM and water content were measured at the start of the experiment and after 3 months. Duplicate samples were dried over P₂O₅/vacuum for 6, 14 and 26 weeks. As can be seen from the figures, drying of the samples to reduce the increase in moisture with time, advantageously increases the FPM and hence the efficacy of the inhaler.

One method employed to overcome moisture increases during the storing of a metered dose inhaler (MDI) uses a plastic tube that has a resealable lid to close the tube. The resealable lid for this tube employs a desiccant to absorb moisture present in the tube. However, such plastic tubes typically increase manufacturing costs and require complex and/or expensive manufacturing processes. The tubes also require a significant amount of storage space relative to the size of the container disposed within the plastic tube.

Another solution is disclosed in PCT application no. PCT/US99/27851 which describes a container storage system wherein a flexible package wraps and seals the pressurised container providing an enclosed volume in which the pressurised container is disposed. The flexible package is impermeable to water vapour whilst being permeable to the propellant, such that the flexible package substantially prevents ingression of water vapour and particulate matter into the enclosed volume while permitting egression of the propellant.

An object of the present invention is to provide a device and method for preventing moisture increase in drug formulations stored in dispensers therefor, which is cost effective and which does not require complex manufacturing processes.

A further object of the present invention is to provide means to reduce or eliminate moisture increases in a dispenser for dispensing a medicament in a fluid propellant.

Accordingly, in one aspect the invention provides a dispenser for dispensing a medicament in a fluid propellant comprising (a) a canister for housing the medicament; (b) a drug dispensing valve made substantially of metal; and (c) moisture absorbing means for absorbing moisture.

Preferably, the moisture absorbing means is a desiccant.

In one aspect, the moisture absorbing means is integral with the valve and/or canister.

In a second aspect, the moisture absorbing means may comprise a component or accessory for use with a canister or valve, wherein the component or accessory is made from a plastics material which is a natural desiccant, such as a polyamide, for example nylon, or may be moulded from other plastics material such as Acetal or PBT and include a desiccant such as a molecular sieve and silica gel.

Typically, the component or accessory takes the form of a cap and/or a seal and/or a lining and/or coating.

Alternatively, or in addition to the first and second aspects described above, the moisture absorbing means may comprise an internal lining or coating of the canister and/or valve. In one embodiment, the moisture absorbing means may be incorporated into a treatment or coating for canisters and/or valves for preventing drug deposition and/or maintaining dose uniformity.

Vapour or moisture absorbing materials suitable for use in the present invention, include desiccants made from inorganic materials such zeolites and aluminas. Such inorganic materials have high water absorption capacities and favourable water absorption isotherm shapes. The water absorption capacity of such materials typically varies from 20 to 50 weight percent.

Other exemplary moisture absorbing materials suitable for use in the present invention include, but are not limited to, alumina, bauxite, anhydrous calcium sulphate, water-absorbing clay, activated bentonite clay, a molecular sieve, or other like materials.

Incorporation of moisture absorbing means into the canister and/or valve according to the invention substantially prevents ingression of water vapour into the canister and absorbs any residual moisture present in the formulation.

In conjunction with the desiccant an additional component may be added to act as a conduit/channelling agent to increase/optimise the efficiency of the moisture absorption properties. Such components may include compounds such as polyethylene glycols.

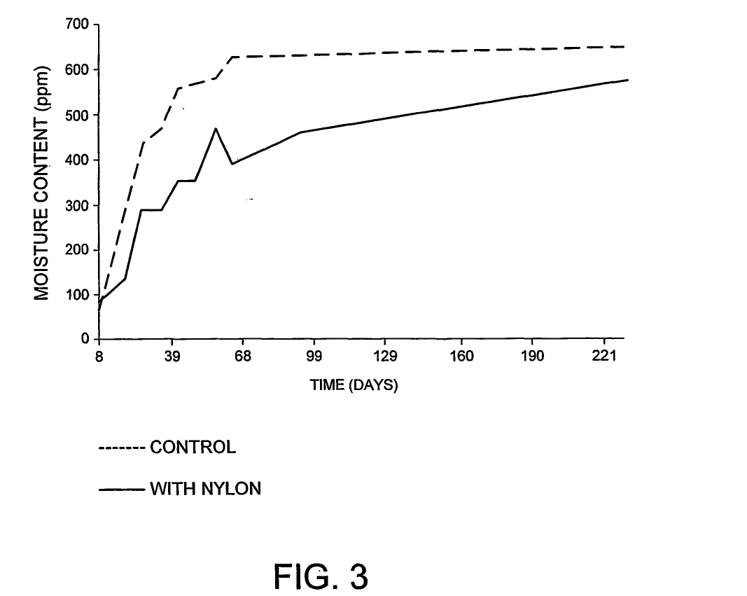

Experiments were conducted on metered dose inhalers having plastic valves with and without the presence of a nylon ring thereon. Nylon is a natural desiccant material. FIG. 3 illustrates how the presence of the nylon ring significantly reduces the increase in moisture in the MDI and thus by inference improves product performance (see Experimental Detail, infra, for methodology).

The desiccant should be present in an amount sufficient to absorb any increases in moisture around the valve area of the MDI and thus alleviate or substantially prevent moisture increases inside the canister.

Typically, 100 μg to 5 g, for example, 1 mg to 1 g, e.g. 100 mg to 500 mg, such as about 100 mg to 250 mg of desiccant may be included.

Typically, the drug-dispensing valve is a drug metering valve.

Preferably, the canister and/or the valve are made of stainless steel or aluminium. The advantages of incorporating a metal drug metering valve and canister include the ability to exert tighter control on component tolerances during manufacture. In addition, studies have found that a conducting component surface that is treated to have a defined surface energy facilitates dose uniformity. Therefore, if the canister and the valve are substantially made of metal or metal alloys, almost the entire MDI can be conducting and contribute towards the maintenance of a consistent dose.

In another aspect, the invention provides a valve made substantially of metal for use in a dispenser for dispensing a medicament in a fluid propellant, the valve having moisture absorbing means for absorbing moisture.

Typically, the drug-dispensing valve is a drug-metering valve.

Preferably, the moisture absorbing means is a desiccant as hereinbefore described.

In another aspect, the invention provides a metered dose inhaler for dispensing a medicament in a fluid propellant comprising a dispenser as defined above and a medicament channelling device, such as an actuator.

In a further aspect, the invention provides a canister for use in a dispenser for dispensing a medicament in a fluid propellant, the canister having moisture absorbing means for absorbing moisture.

Preferably, the moisture absorbing means is a desiccant as hereinbefore described.

The moisture absorbing means takes the form of a crimped cap, and/or coating, and/or treatment, and/or lining, and/or other accessory for sealing the canister. The moisture absorbing means may be made of a material which is naturally a desiccant or a plastics material including a desiccant.

Typically, the canister contains a pharmaceutical aerosol formulation comprising a medicament and a fluorocarbon propellant.

In a further aspect, the invention provides a method of preventing moisture increase in a dispenser for dispensing a medicament in a fluid propellant the dispenser comprising (a) a canister for housing the medicament; (b) a drug-dispensing valve made substantially of metal, the method comprising the step of including moisture absorbing means for absorbing moisture.

In still another aspect, the invention provides a method of preventing moisture increase in a dispenser for dispensing a medicament in a fluid propellant having a canister for housing the medicament and a drug-dispensing valve, the method comprising the use of a canister and/or a drug metering valve as defined above.

Typically, the valve is a drug-metering valve.

Preferably, the moisture absorbing means is a desiccant.

The moisture absorbing means may comprise a component or valve accessory which is made from a plastics material which is a natural desiccant, such as nylon, or may be moulded from other plastics material such as acetal or PBT and include a desiccant such as a molecular sieve and silica gel. Other vapour or moisture absorbing materials include desiccants made from inorganic materials such zeolites and aluminas. Such inorganic materials have high water absorption capacities and favourable water absorption isotherm shapes. The water absorption capacity of such materials typically varies from 20 to 50 weight percent.

Other exemplary moisture absorbing materials include, but are not limited to, alumina, bauxite, anhydrous, calcium sulphate, water-absorbing clay, activated bentonite day, a molecular sieve, or other like materials. The invention is designed to substantially prevent ingression of water vapour into the canister and absorbs any residual moisture present in the formulation.

In conjunction with the desiccant an additional compound may be added to act as a conduit/channelling agent to increase/optimise the efficiency of the moisture absorption properties. Such materials may include compounds such as polyethylene glycols.

Typically, the component or accessory takes the form of a cap and/or a seal and/or a lining and/or a coating and/or a treatment.

The metered dose inhalers may be prepared by methods of the art (e.g. see Byron above and U.S. Pat. No. 5,345,980).

Conventionally, the canisters and caps for use in MDI's are made of aluminium or an alloy of aluminium although other metals not affected by the drug formulation, such as stainless steel, an alloy of copper, or tin plate, may be used. An MDI canister may also be fabricated from glass or plastics. Preferably, however, the MDI canisters and caps employed in the present invention are made of aluminium or an alloy thereof or stainless steel.

The MDI comprises a pressurised container having a vial with a valve disposed therein. While the pressurised container preferably includes a metering valve, other valve systems are not beyond the scope of the present invention. Other valve systems include, but are not limited to, wedge gate valve systems, double-disc gate valve systems, globe and angle valve systems, swing check valve systems, end cock valve systems, and other like valve systems. Since the pressurised container is preferably part of an MDI, the valve design is typically a function of providing a predetermined dosage or amount of the drug contained within the pressurised container to a user.

The drug-metering valve may consist of parts substantially made of metal, e.g. stainless steel. Additionally, seals and “O” rings of various materials (e.g., nitrile rubbers, polyurethane, acetyl resin, fluorocarbon polymers), or other elastomeric materials are employed in and around the valve.

The valve typically comprises a valve body having an inlet port through which the pharmaceutical aerosol formulation may enter said valve body, an outlet port through which the pharmaceutical aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

The valve may be a metering valve in which the valve body has a metering chamber, a sampling chamber and therebetween a second sealing ring within which the stem is slidably movable, the valve stem having a transfer passage such that in the valve-closed position the dispensing passage is isolated from the metering chamber and the metering chamber is in communication with the sampling chamber via the transfer passage, and in the valve-open position the dispensing passage is in communication with the metering chamber and the transfer passage is isolated from the metering-chamber. The metering volumes are typically from 50 to 100 μl, such as 50 μl or 63 μl.

The sealing ring may be formed by cutting a ring from a sheet of suitable material. Alternatively, the sealing ring may be formed by a moulding process such as an injection moulding, a compression moulding or a transfer moulding process.

Typically, the sealing ring and/or second sealing ring comprise an elastomeric material. The ring is typically resiliently deformable.

The elastomeric material may either comprise a thermoplastic elastomer (TPE) or a thermoset elastomer, which may optionally be cross-linked. The sealing ring may also comprise a thermoplastic elastomer blend or alloy in which an elastomeric material is dispersed in a thermoplastic matrix. The elastomers may optionally additionally contain conventional polymer additives such as processing aids, colorants, tackifiers, lubricants, silica, talc, or processing oils such as mineral oil in suitable amounts.

Suitable thermoset rubbers include butyl rubbers, chloro-butyl rubbers, bromo-butyl rubbers, nitrile rubbers, silicone rubbers, fluorosilicone rubbers, fluorocarbon rubbers, polysulphide rubbers, polypropylene oxide rubbers, isoprene rubbers, isoprene-isobutene rubbers, isobutylene rubbers or neoprene (polychloroprene) rubbers.

Suitable thermoplastic elastomers comprise a copolymer of about 80 to about 95 mole percent ethylene and a total of about 5 to about 20 mole percent of one or more comonomers selected from the group consisting of 1-butene, 1-hexene, and 1-octene as known in the art. Two or more such copolymers may be blended together to form a thermoplastic polymer blend.

Another suitable class of thermoplastic elastomers are the styrene-ethylene/butylene-styrene block copolymers. These copolymers may additionally comprise a polyolefin (e.g. polypropylene) and a siloxane.

Thermoplastic elastomeric material may also be selected from one or more of the following: polyester rubbers, polyurethane rubbers, ethylene vinyl acetate rubber, styrene butadiene rubber, copolyether ester TPE, olefinic TPE, polyester amide TPE and polyether amide TPE.

Other suitable elastomers include ethylene propylene diene rubber (EPDM). The EPDM may be present on its own or present as part of a thermoplastic elastomer blend or alloy, e.g. in the form of particles substantially uniformly dispersed in a continuous thermoplastic matrix (e.g. polypropylene or polyethylene). Commercially available thermoplastic elastomer blend and alloys include the SANTOPRENE™ elastomers. Other suitable thermoplastic elastomer blends include butyl-polyethylene (e.g. in a ratio ranging between about 2:3 and about 3:2) and butyl-polypropylene.

Any parts of the valve which contact the pharmaceutical aerosol suspension may be coated with materials such as fluoropolymer materials which reduce the tendency of medicament to adhere thereto. Suitable fluoropolymers include polytetrafluoroethylene (PTFE) and fluoroethylene propylene (FEP). Any movable parts may also have coatings applied thereto, which enhance their desired movement characteristics. Frictional coatings may therefore be applied to enhance frictional contact and lubricants used to reduce frictional contact as necessary.

Typically, the sealing ring and/or the second sealing ring additionally comprises lubricant material. Suitably, the sealing ring and/or the second sealing ring comprises up to 30%, preferably from 5 to 20% lubricant material.

In addition, the stem may also comprise lubricant material. Suitably, the valve stem comprises up to 30%, preferably from 5 to 20% lubricant material.

The term ‘lubricant’ herein means any material, which reduces friction between the valve stem and seal. Suitable lubricants include silicone oil or a fluorocarbon polymer such as polytetrafluoroethane (PTFE) or fluoroethylene propylene (FEP).

Lubricant can be applied to the stem, sealing ring or a second sealing ring by any suitable process including coating and impregnation, such as by injection or a tamponage process.

In medical use the canisters in accordance with the invention contain a pharmaceutical aerosol formulation comprising a medicament and a fluorocarbon or hydrogen-containing chlorofluorocarbon propellant.

Suitable propellants include, for example, C₁₋₄hydrogen-containing chlorofluorocarbons such as CH₂ClF, CClF₂CHClF, CF₃CHClF, CHF₂CClF₂, CHClFCHF₂, CF₃CH₂Cl and CClF₂CH₃; C₁₋₄hydrogen-containing fluorocarbons such as CHF₂CHF₂, CF₃CH₂F, CHF₂CH₃ and CF₃CHFCF₃; and perfluorocarbons such as CF₃CF₃ and CF₃CF₂CF₃.

Where mixtures of the fluorocarbons or hydrogen-containing chlorofluorocarbons are employed they may be mixtures of the above identified compounds or mixtures, preferably binary mixtures, with other fluorocarbons or hydrogen-containing chlorofluorocarbons for example CHClF₂, CH₂F₂ and CF₃CH₃. Preferably a single fluorocarbon or hydrogen-containing chlorofluorocarbon is employed as the propellant. Particularly preferred as propellants are C₁₋₄hydrogen-containing fluorocarbons such as 1,1,1,2-tetrafluoroethane (CF₃CH₂F) and 1,1,1,2,3,3,3-heptafluoro-n-propane (CF₃CHFCF₃) or mixtures thereof.

The pharmaceutical formulations for use in the canisters of the invention suitably contain no components which provoke the degradation of stratospheric ozone. In particular the formulations are preferably substantially free of chlorofluorocarbons such as CCl₃F, CCl₂F₂ and CF₃CCl₃.

The propellant may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations which are free or substantially free of volatile adjuvants are preferred. In certain cases, it may be desirable to include appropriate amounts of water, which can be advantageous in modifying the dielectric properties of the propellant.

The invention is particularly useful with propellants (including propellant mixtures) which are more hygroscopic than P11, P114 and/or P12 such as HFA-134a and HFA-227.

A polar co-solvent such as C₂₋₆ aliphatic alcohols and polyols e.g. ethanol, isopropanol and propylene glycol, preferably ethanol, may be included in the drug formulation in the desired amount to improve the dispersion of the formulation, either as the only excipient or in addition to other excipients such as surfactants. Suitably, the drug formulation may contain 0.01 to 5% w/w based on the propellant of a polar co-solvent e.g. ethanol, preferably 0.1 to 5% w/w e.g. about 0.1 to 1% w/w.

A surfactant may also be employed in the aerosol formulation. Examples of conventional surfactants are disclosed in EP-A-372,777. The amount of surfactant employed is desirable in the range 0.0001% to 50% weight to weight ratio relative to the medicament, in particular, 0.05 to 5% weight to weight ratio. Preferred surfactants are lecithin, oleic acid and sorbitan trioleate. Preferred formulations, however, are free or substantially free of surfactant.

Pharmaceutical formulations may contain 0.0001 to 50% w/w, preferably 0.001 to 20%, for example 0.001 to 1% of sugar relative to the total weight of the formulation. Generally the ratio of medicament to sugar falls within the range of 1:0.01 to 1:100 preferably 1:0.1 to 1:10. Typical sugars which may be used in the formulations include, for example, sucrose, lactose and dextrose, preferably lactose, and reducing sugars such as mannitol and sorbitol, and may be in micronised or milled form.

The final aerosol formulation desirably contains 0.005-10% w/w, preferably 0.005 to 5% w/w, especially 0.01 to 1.0% w/w, of medicament relative to the total weight of the formulation.

Medicaments which may be administered in the aerosol formulations include any drug useful in inhalation therapy. Appropriate medicaments may thus be selected from, for example, analgesics, e.g. codeine, dihydromorphine, ergotamine, fentanyl or morphine; anginal preparations, e.g. diltiazem; antiallergics, e.g. cromoglycate, ketotfen or nedocromil; antiinfectives e.g. cephalosporins, penicillins, streptomycin, sulphonamides, tetracyclines and pentamidine; antihistamines, e.g. methapyrilene; anti-inflammatories, e.g. beclomethasone, flunisolide, budesonide, tipredane, triamcinolone acetonide, fluticasone or mometasone; antitussives, e.g. noscapine; bronchodilators, e.g. ephedrine, epinephrine, fenoterol, formoterol, isoprenaline, metaproterenol, phenylephrine, phenylpropanolamine, pirbuterol, reproterol, rimiterol, salbutamol, terbutaline, isoetharine, tulobuterol, 4-hydroxy-7-[2-[[2-[[3-(2phenylethoxy)propyl]sulfonyl]amino]ethyl-2(3H)-benzothiazolone; orciprenaline or (−)-4-amino-3,4-dichloro-α-[[[6-[2-(2-pyridinyl)ethoxy]hexyl]amino]methyl]-benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon. It will be clear to a person skilled in the art that, where appropriate, the medicaments may be used in the form of salts (e.g. as alkali metal or amine salts or as acid addition salts) or as esters (e.g. lower alkyl esters) or as solvates (e.g. hydrates) to optimise the activity and/or stability of the medicament and/or to minimise the solubility of the medicament in the propellant. It will further be clear to a person skilled in the art that where appropriate, the medicaments may be used in the form of a pure isomer, for example, R-salbutamol or RR formotenol.

Particularly preferred medicaments for administration using aerosol formulations in accordance with the invention include anti-allergics, bronchodilators and anti-inflammatory steroids of use in the treatment of respiratory disorders such as asthma by inhalation therapy, for example cromoglycate (e.g. as the sodium salt), salbutamol (e.g. as the free base or the sulphate salt), salmeterol, formoterol (e.g. as the fumarate salt), terbutaline (e.g. as the sulphate salt), reproterol (e.g. as the hydrochloride salt), a beclomethasone ester (e.g. the diproprionate), a fluticasone ester (e.g. the propionate). Salmeterol, salbutamol, fluticasone propionate, beclomethasone dipropionate and physiologically acceptable salts and solvates thereof are especially preferred. Preferably, if the medicament is salmeterol, it is not as the xinafoate salt.

It will be appreciated by those skilled in the art that the aerosol formulations according to the invention may, if desired, contain a combination of two or more active ingredients. Aerosol compositions containing two active ingredients are known for the treatment of respiratory disorders such as asthma, for example, formoterol and budesonide, salmeterol (e.g. as the xinafoate salt) and fluticasone (e.g. as the propionate ester), salbutamol and beclomethasone (as the dipropionate ester) are preferred.

Particularly preferred formulations for use in the canisters of the present invention comprise a medicament and a C₁₋₄ hydrofluoroalkane particularly 1,1,1,2-tetrafluoroethane and 1,1,1,2,3,3,3-n-heptafluoropropane or a mixture thereof as propellant.

Preferred formulations are free or substantially free of formulation excipients. Thus, preferred formulations consist essentially of (or consist of) the medicament and the selected propellant.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The particulate medicament is added to a charge vessel and liquefied propellant is pressure filled through the charge vessel into a manufacturing vessel. The drug suspension is mixed before re-circulation to a filling machine and an aliquot of the drug suspension is then filled through the metering valve into the canister. Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler for administration of the medicament into the lungs or nasal cavity of a patient. Suitable channelling devices comprise for example a valve actuator and a cylindrical or cone-like passage through which medicament may be delivered from the filled canister via the metering valve to the nose or mouth of a patient e.g. a mouthpiece actuator. Metered dose inhalers are designed to deliver a fixed unit dosage of medicament per actuation or “puff”, for example in the range of 10 to 5000 microgram medicament per puff.

Administration of medicament may be indicated for the treatment of mild, moderate or severe acute or chronic symptoms or for prophylactic treatment. It will be appreciated that the precise dose administered will depend on the age and condition of the patent, the particular particulate medicament used and the frequency of administration and will ultimately be at the discretion of the attendant physician. When combinations of medicaments are employed the dose of each component of the combination will in general be that employed for each component when used alone. Typically, administration may be one or more times, for example from 1 to 8 times per day, giving for example 1,2,3 or 4 puffs each time. Each valve actuation, for example, may deliver 5 μg, 50 μg, 100 μg, 200 μg or 250 μg of a medicament.

Typically, each filled canister for use in a metered dose inhaler contains 60, 100, 120 or 200 metered doses or puffs of medicament; the dosage of each medicament is either known or readily ascertainable by those skilled in the art.

A still further aspect of the present invention comprises a method of treating respiratory disorders such as, for example, asthma, which comprises administration by inhalation of an effective amount of an aerosol formulation as herein described from a metered dose inhaler of the present invention.

It will be understood that the present disclosure is for the purpose of illustration only and the invention extends to modifications, variations and improvements thereto which will be within the ordinary skill of the person skilled in the art.

### EXPERIMENTAL DETAIL

### 1. Method for the Determination of the Water Content of Metered Dose Inhalers using a Mitsubishi Moisturemeter

A Mitsubishi Moisturemeter was used to calculate the water content of the canisters in ppm (μg/g).

The moisturemeter is first calibrated to ensure the accuracy and precision of the water determination, i.e. there must be less than a relative standard deviation of less than 3%. The sample procedure involves weighing of the canister to four decimal places. The canister is linked to the moisturemeter and ten actuations at approximately 2 second intervals are discharged into the apparatus. The meter will give a reading of the water content of the actuations in μg. The canister is subsequently reweighed.

The water content is calculated using the following equation:

$$Water content ( ppm ) = \frac{meter reading ( µ g )}{weight of discharged actuations ( g )}$$

### 2. Determination of the Fine Particle Mass (FPM) in Salmeterol Metered Dose Inhalers 25 μg by Cascade Impaction.

The method involves the use of the Andersen Cascade Impactor in the characterisation of fine drug particles emitted from Salmeterol inhalers by their aerodynamic size distribution. During sample preparation the required number of actuations must be discharged under the following controlled environmental conditions: temperature 17 to 23° C.; relative humidity 45 to 55%. The temperature and the relative humidity must be measured before and after the required number of actuations are discharged. The drug substance discharged is collected and deposited from each stage of the cascade impactor in methanol. For example, a method for the collection of drug deposited on each stage of the cascade impactor is presented below.

Place the canisters in clean dry actuators and discharge four actuations to waste in order to prime the inhaler. Between each of the four priming actuations shake the inhaler for not less than five seconds. Record the weight of the inhaler.

Assemble the impactor, shake the inhaler and deliver one actuation. Wait 30 seconds and repeat until 10 actuations have been discharged into the cascade impactor. Record the weight of the inhaler. Transfer the deposited drug from all apparatus contacted using methanol and measure quantitatively using HPLC. Thus, the Salmeterol content of each sample solution can be determined in micrograms. is The FPM is determined from the total deposition of Salmeterol per actuation.

$$µ g per actuation = \frac{MRF \times Au \times Dfu \times CF \times 1000}{N}$$

Where:

Au=Area of sample peak (HPLC)

MRF=Mean response factor calculated for standard injections

Dfu=Dilution factor for the sample solution

N=Number of actuations

CF=salt to base conversion factor for Salmeterol

1. A dispenser for dispensing a medicament in a fluid propellant comprising (a) a canister for housing the medicament; (b) a drug-dispensing valve made substantially of metal; and (c) moisture absorbing means for absorbing moisture.

2. A dispenser as claimed in claim 1, wherein the moisture absorbing means takes the form of a desiccant.

3. A dispenser as claimed in any one of claims 1 or 2, wherein the moisture absorbing means is integral with the valve and/or canister.

4. A dispenser as claimed in any one of claims 1 or 2, wherein the moisture absorbing means comprises a component or accessory for use with the canister and/or valve, wherein the component or accessory is made from a plastics material that is a desiccant.

5. A dispenser as claimed in claim 4, wherein the desiccant is a polyamide.

6. A dispenser as claimed in any one of claims 1 or 2, wherein the moisture absorbing means comprises a component or accessory for use with a canister and/or valve, the component or accessory being made from a plastics material that includes a desiccant.

7. A dispenser as claimed in claim 6, wherein the component and/or accessory is made from acetal and/or PBT and a desiccant.

8. A dispenser as claimed in claim 6 or 7, wherein the desiccant is selected from the group consisting of a silica gel desiccant; a zeolite; an alumina; a bauxite; anhydrous calcium sulphate; water-absorbing clay; activated bentonite clay; a molecular sieve; and any mixtures thereof.

9. A dispenser as claimed in any one of claims 4 to 8 wherein the component or accessory takes the form of a cap and/or seal and/or lining and/or coating.

10. A dispenser as claimed in any one of the preceding claims, wherein the moisture absorbing means comprises an internal lining or coating of the canister and/or valve.

11. A dispenser as claimed in any one of the preceding claims wherein the moisture absorbing means is incorporated into a canister and/or valve treatment or coating for preventing drug deposition and/or maintaining dose uniformity.

12. A dispenser as claimed in any one of claims 10 or 11, wherein the dessicant is selected from the group consisting of a silica gel desiccant; a zeolite; an alumina; a bauxite; anhydrous calcium sulphate; water-absorbing clay; a molecular sieve; and any mixtures thereof.

13. A dispenser as claimed in any one of the preceding claims, wherein the moisture absorbing means comprises 100 μg to 5 g of desiccant.

14. A dispenser as claimed in claim 13, wherein the moisture absorbing means comprises 1 mg to 1 g of desiccant.

15. A dispenser as claimed in claim 14, wherein the moisture absorbing means comprises 100 mg to 500 mg of desiccant.

16. A dispenser as claimed in claim 15, wherein the moisture absorbing means comprises 100 mg to 250 mg of desiccant.

17. A dispenser as claimed in any one of the preceding claims, wherein the moisture absorbing means includes a conduit/channelling agent to increase/optimise the efficiency of the moisture absorption properties.

18. A dispenser as claimed in claim 17, wherein the conduit/channelling agent is a polyethylene glycol.

19. A dispenser as claimed in any one of the preceding claims, wherein the moisture absorbing means reduces the rise in moisture content over time, and/or reduces the decrease in Fine Particulate Mass (FPM) over time by between 20 and 100%.

20. A dispenser as claimed in claim 19, wherein the moisture absorbing means reduces the rise in moisture content over time, and/or reduces the decrease in Fine Particulate Mass (FPM) over time by between 40 to 70%.

21. A dispenser as claimed in claim 20, wherein the moisture absorbing means reduces the rise in moisture content over time, and/or reduces the decrease in Fine Particulate Mass (FPM) over time by between 45 and 55%.

22. A dispenser as claimed in any one of the preceding claims, comprising a medicament in a fluid propellant, wherein the fluid propellant includes a hydrofluoroalkane.

23. A dispenser as claimed in claim 22, wherein the hydrofluoroalkane is selected from the group consisting of 1,1,1,2-tetrafluorethane, 1,1,1,2,3,3,3-heptafluoropropane; and any mixtures thereof.

24. A dispenser as claimed in any one of the preceding claims, wherein the valve is a drug-metering valve.

25. A dispenser as claimed in any one of the preceding claims, wherein the canister and/or the valve are made of stainless steel.

26. A dispenser as claimed in any one of claims 1 to 24, wherein the canister and/or the valve are made of aluminium.

27. A valve made substantially of metal for use in a dispenser for dispensing a medicament in a fluid propellant, the valve having moisture absorbing means for absorbing moisture.

28. A valve as claimed in claim 27 that is a drug-metering valve.

29. A valve as claimed in claim 27 or claim 28 wherein the moisture absorbing means is a desiccant.

30. A metered dose inhaler comprising a dispenser according to any one of claims 1 to 26 and a medicament channelling device.

31. A metered dose inhaler as claimed in claim 30, wherein the medicament channelling device is an actuator.

32. A canister for use in a dispenser for dispensing a medicament in a fluid propellant, the canister having moisture absorbing means for absorbing moisture.

33. A canister as claimed in claim 32 wherein the moisture absorbing means takes the form of a desiccant.

34. A canister as claimed in claim 32 or claim 33 wherein the moisture absorbing means takes the form of a crimped cap, and/or coating, and/or treatment and/or lining and/or other accessory for sealing the canister.

35. A canister as claimed in claim 34 wherein the cap and/or coating, and/or treatment and/or lining and/or other accessory is made of a material that is naturally a desiccant or a plastics material including a desiccant.

36. A canister as claimed in any one of claims 32 to 35 further comprising a pharmaceutical aerosol formulation comprising a medicament and a fluorocarbon propellant.

37. A method of preventing moisture increase in a dispenser for dispensing a medicament in a fluid propellant, the dispenser comprising (a) a canister for housing the medicament; and (b) a drug dispensing valve made substantially of metal, the method comprising the step of including moisture absorbing means for absorbing moisture therein.

38. A method of preventing moisture increase in a dispenser for dispensing a medicament in a fluid propellant having a canister for housing the medicament and a drug dispensing valve, the method comprising the use of a canister as claimed in any one of claims 32 to 36 and/or a drug-dispensing valve as claimed in any one of claims 27 to 29.

39. A method as claimed in any one of claims 37 and 38, wherein the valve is a drug-metering valve.

40. A method as claimed in any one of claims 37 to 39, wherein the moisture absorbing means is a desiccant.

41. A method of treating respiratory disorders which comprises administration by inhalation of an effective amount of a medicament from a dispenser as claimed in any one of claims 1 to 26.

42. A dispenser substantially as described with reference to the accompanying description and drawings.

43. A canister for use in a dispenser for dispensing a medicament in a fluid propellant substantially as described in the accompanying description and drawings.

44. A drug-dispensing valve substantially as described in the accompanying description and drawings.

45. A method of preventing moisture increase in a dispenser for dispensing a medicament in a fluid propellant having a canister for housing the medicament and a drug-dispensing valve substantially as described in the accompanying description and drawings.
